(19) United States
(12) Patent Application Publication
(10) Pub. No.: US 2023/0181918 A1
(43) Pub. Date: Jun. 15, 2023

(54) TRANSDUCER ARRAY WITH ADHESIVE LAYER SHAPED TO REDUCE SKIN IRRITATION

(71) Applicant: Novocure GmbH, Root D4 (CH)

(72) Inventors: Boaz MARSAULT, Haifa (IL); Elie YAACOBI, Haifa (IL)

(73) Assignee: Novocure GmbH, Root D4 (CH)

(21) Appl. No.: 18/080,026

(22) Filed: Dec. 13, 2022

Related U.S. Application Data
(60) Provisional application No. 63/289,508, filed on Dec. 14, 2021.

(51) Int. Cl. A61N 1/40; A61N 1/04

(52) U.S. Cl. CPC A61N 1/40; A61N 1/0476; A61N 1/0492

(57) ABSTRACT

A transducer apparatus for delivering tumor treating fields to a subject's body, the transducer apparatus including: an array of electrodes configured to be positioned over the subject's body with a face of the array facing the subject's body; a substrate including an adhesive layer, wherein the array of electrodes is disposed entirely on a first side of the substrate, the face of the array faces away from the substrate, the adhesive layer is on the first side of the substrate facing the same direction as the face of the array; and, when viewed from a direction perpendicular to the face of the array, a non-adhesive region is located between a pair of adjacent electrodes of the array, the non-adhesive region spanning at least 25% of a total distance between the pair of adjacent electrodes for at least one measurement measured along a straight line between the pair of adjacent electrodes.

### CROSS REFERENCE TO RELATED APPLICATIONS

This application claims priority to U.S. Provisional Patent Application No. 63/289,508, filed Dec. 14, 2021, which is hereby incorporated by reference in its entirety.

### BACKGROUND

Tumor treating fields (TTFields) are low intensity alternating electric fields within the intermediate frequency range, which may be used to treat tumors as described in U.S. Pat. No. 7,565,205. TTFields are induced non-invasively into a region of interest by applying AC voltages between transducers placed on the patient's body. Conventionally, transducers used to generate TTFields include a plurality of ceramic disks. One side of each ceramic disk is positioned against the patient's skin, and the other side of each disc has a conductive backing. Electrical signals are applied to this conductive backing, and these signals are capacitively coupled into the patient's body through the ceramic discs. Conventional transducer designs include arrays of ceramic disks attached to the subject's body via an adhesive bandage.

### SUMMARY OF THE INVENTION

One aspect of the invention is directed to a transducer apparatus for delivering tumor treating fields to a subject's body, the transducer apparatus including: an array of electrodes, the array configured to be positioned over the subject's body with a face of the array facing the subject's body; a substrate comprising an adhesive layer for attaching the transducer apparatus to the subject's body, wherein: the array of electrodes is disposed entirely on a first side of the substrate, the face of the array faces away from the substrate, the adhesive layer is on the first side of the substrate facing the same direction as the face of the array; and wherein, when viewed from a direction perpendicular to the face of the array, a non-adhesive region, where no adhesive layer is present, is located between a pair of adjacent electrodes of the array, wherein the non-adhesive region spans at least 25% of a total distance between the pair of adjacent electrodes for at least one measurement as measured along a straight line between the pair of adjacent electrodes.

The above aspect of the invention is exemplary, and other aspects and variations of the invention will be apparent from the following detailed description of embodiments.

### BRIEF DESCRIPTION OF THE DRAWINGS

FIG. 1 depicts an example of transducers located on a subject's head.

FIG. 2 depicts an example of transducers located on a subject's body.

FIGS. 3A-3F depict top views and cutaway views of example structures of transducers.

FIGS. 4-10 depict example layouts of arrays of electrodes and non-adhesive regions on a transducer apparatus.

FIG. 11 is a flowchart depicting an example of applying TTFields to a subject's body.

### DESCRIPTION OF EMBODIMENTS

This application describes exemplary transducer apparatuses used to apply TTFields to a subject's body for treating one or more cancers. This application also describes exemplary methods to apply TTFields to a subject's body using transducers.

Transducers used to apply TTFields to a subject's body often include multiple electrodes coupled together on a substrate and attached to the subject's body at a desired location via an adhesive layer (e.g., an adhesive backing of the substrate or separately applied adhesive). Conventional transducers use an adhesive layer that is relatively large in surface area to hold the electrodes against the subject's skin for a desired time period during application of TTFields to the subject's body. However, subjects can experience skin irritation on portions of their skin that are contacted by the adhesive layer during treatment.

The inventors have now recognized that a need exists for transducers that use a relatively small amount of adhesive to attach the transducer to the subject's body while being capable of maintaining the transducer in place on the subject's body for a desired time period (e.g., up to 3 days). Reducing the amount of adhesive in contact with the subject's body may reduce or minimize irritation of the subject's skin. In addition, the inventors have now recognized that a need exists for transducers that can be shifted to reduce, minimize, or prevent skin irritation without significantly changing the field intensity of TTFields being induced in the subject's body. In particular, transducers that are able to be shifted so that skin previously contacted by an adhesive layer can be uncovered without substantially moving the transducer from an optimal location on the subject's body are desired. The shifting of the transducer apparatuses can reduce, minimize, or prevent skin irritation while maintaining the transducer in an optimal or near optimal location on the subject's body. As such, the transducers can continuously induce TTFields at a desired location and power level to target a region of interest (e.g., tumor) in the subject's body, thereby improving patient outcomes.

Disclosed transducer apparatuses may have an array of electrodes, a substrate with an adhesive layer for attaching the transducer to a subject's body, and at least one non-adhesive region, where no adhesive layer is present, located between a pair of electrodes of the array. The transducer apparatus can be shifted via rotation or translation of the array of electrodes so that one or more portions of the subject's skin that were previously contacted by the adhesive layer can be uncovered, while maintaining an optimal or near optimal location of the transducer on the subject's body. The disclosed transducer apparatuses may include an electrode array that can be attached to the subject's skin using a smaller amount of adhesive than was previously required.

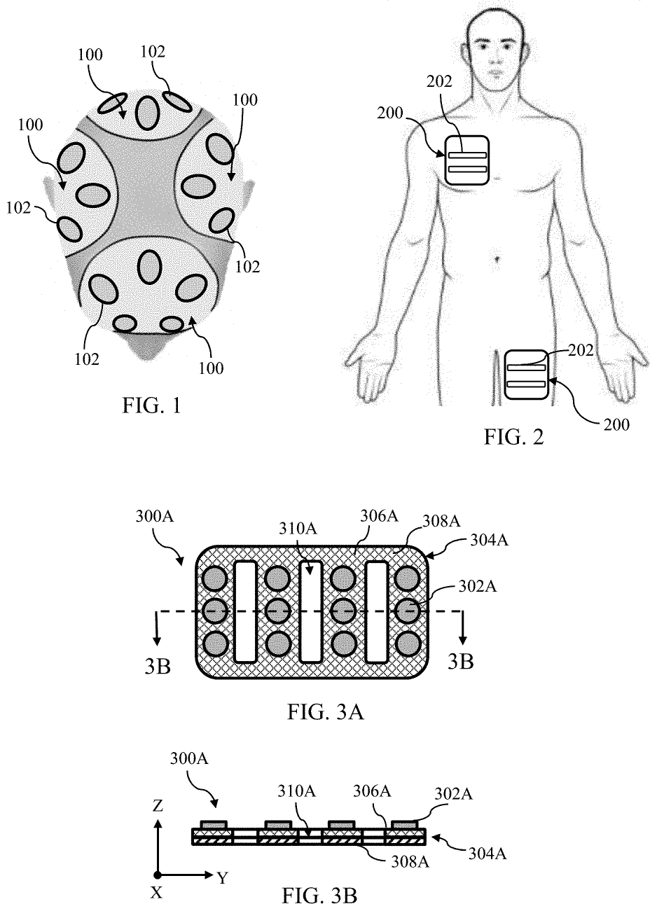

FIG. 1 depicts transducers 100 positioned on the head of a subject's body. Such an arrangement of transducers 100 on a subject's head is capable of applying TTFields to a tumor in a region of the subject's brain. Various other positions and/or orientations on the subject's head may be selected for placement of transducers. Each transducer 100 may have an array of electrodes disposed thereon. Each transducer 100 may be placed on a subject's head with a face of the array of electrodes facing and conforming to the subject's head. As illustrated, the transducers 100 on the subject's head may include non-adhesive regions 102 (e.g., in the form of cutouts) at various locations on the transducers 100. FIG. 2 depicts transducers 200 attached to other portions (e.g., a thorax/torso and a thigh) of the subject's body. The transducers 200 may be affixed to the subject's body via an adhesive layer of the transducers 200. Each transducer 200 may be placed over the subject's body with a face of the array of electrodes facing and conforming to the subject's body. As illustrated, the transducers 200 on the subject's body may include non-adhesive regions 202 (e.g., in the form of cutouts) at various locations.

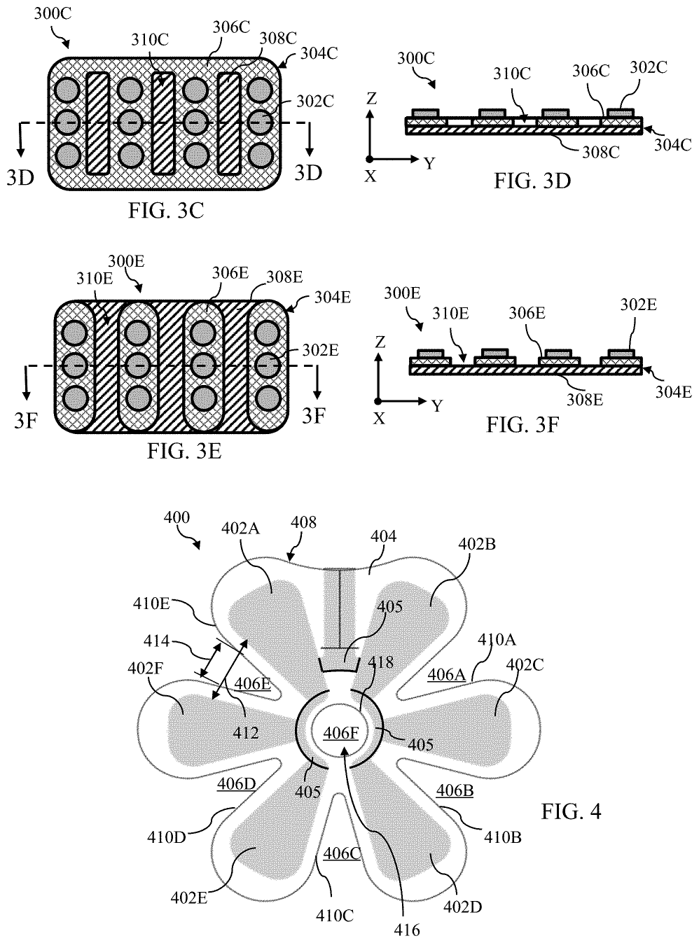

The structure of transducers having adhesive layers and non-adhesive regions may take many forms. FIGS. 3A, 3C, and 3E illustrate top views of example transducers, with FIGS. 3B, 3D, and 3F illustrating cutaway views of the example transducers of FIGS. 3A, 3C, and 3E, respectively. In FIGS. 3B, 3D, and 3F, the cross hatching is on the sectioned portions. In FIGS. 3B and 3D, the lines between the cross-hatched sections represent sidewalls, or one or more portions of the substrate (304A, 304C) that are continuous beyond the plane of the page (e.g., in the negative X-direction), showing the one or more layers of the substrate (304A, 304C) as being connected in a plane beyond the cross section taken through non-adhesive regions.

In each of FIGS. 3A-3F, the transducer (300A, 300C, 300E) has a plurality of electrodes (302A, 302C, 302E) positioned on a substrate (304A, 304C, 304E). The transducer (300A, 300C, 300E) may comprise an array of substantially flat electrodes (302A, 302C, 302E). The array of electrodes may be capacitively coupled. In one example, the electrodes (302A, 302C, 302E) are non-ceramic dielectric materials positioned over a plurality of flat conductors such as, for example, polymer films disposed over pads on a printed circuit board or over flat pieces of metal. In another example, the electrodes (302A, 302C, 302E) are ceramic electrodes connected via conductive wires.

The substrate (304A, 304C, 304E) comprises an adhesive layer (306A, 306C, 306E) for attaching the transducer to a subject's body. The adhesive layer (306A, 306C, 306E) may be any desired adhesive compatible with human skin and/or a conductive medical gel. The substrate (304A, 304C, 304E) may include a structural backing (308A, 308C, 308E) including, for example, cloth, foam, and/or flexible plastic. This backing (308A, 308C, 308E) may function as a bandage having the adhesive layer (306A, 306C, 306E) thereon. The transducer (300A, 300C, 300E) includes at least one non-adhesive region (310A, 310C, 310E), where no adhesive layer is present, located between two adjacent electrodes (302A, 302C, 302E).

As illustrated, the array of electrodes (302A, 302C, 302E) is disposed entirely on a first side (e.g., top) of the substrate (304A, 304C, 304E), and the face of the array of electrodes (302A, 302C, 302E) faces away from the substrate (304A, 304C, 304E) (e.g., facing the +Z direction). The adhesive layer (306A, 306C, 306E) is on the same first side (top) of the substrate (304A, 304C, 304E) and faces the same direction (+Z direction) as the face of the array.

In FIGS. 3A and 3B, the non-adhesive region 310A is a space where no portion of the substrate 304A is located. That is, the non-adhesive regions 310A are cutouts that extend through both the adhesive layer 306A and the backing 308A of the substrate 304A. In some embodiments where the non-adhesive regions 310A are cutouts, the adhesive layer 306A need not be located directly between the backing 308A and the electrodes 302A. Instead, the electrodes 302A may be directly mounted to the backing 308A, and the adhesive layer 306A may be applied as conductive gel to all exposed areas on the backing 308A surrounding the electrodes 302A. In FIGS. 3C-3F, the non-adhesive region (310C, 310E) is a region of the substrate that does not have the adhesive layer. For example, as shown in FIGS. 3D and 3F, the non-adhesive region (310C, 310E) may be a space on the +Z direction facing the surface of the backing (308C, 308E) where no layer (e.g., adhesive or otherwise) is located. In another example, the non-adhesive region (310C, 310E) may include a layer of non-adhesive material interposed between the different portions of the adhesive layer (306C, 306E). In FIGS. 3A-3D, the transducer (300A, 300C) may include one continuous adhesive layer (306A, 306C). In FIGS. 3E and 3F, the transducer 300E may include multiple adhesive layers 306E separated by the non-adhesive regions 310E. Other relative arrangements of the substrate, adhesive layer(s), and non-adhesive regions may be used in other embodiments.

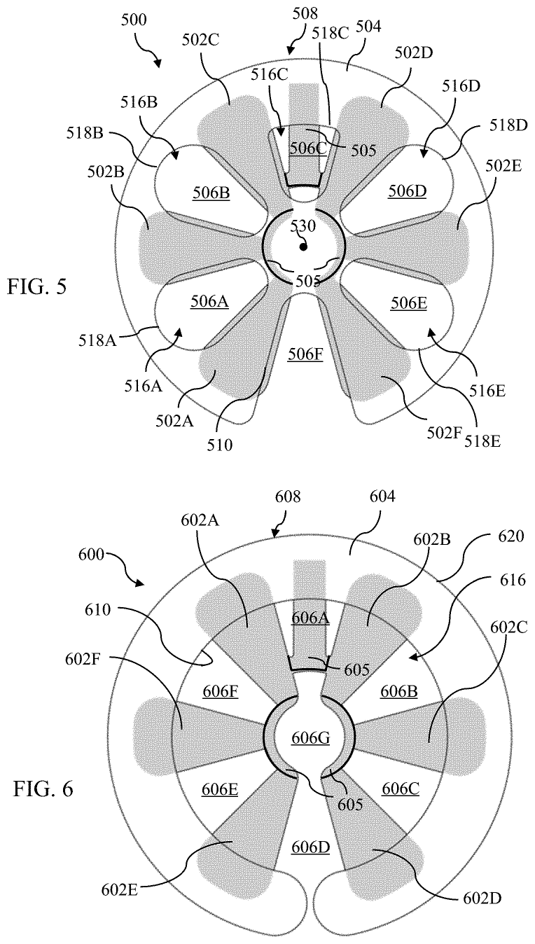

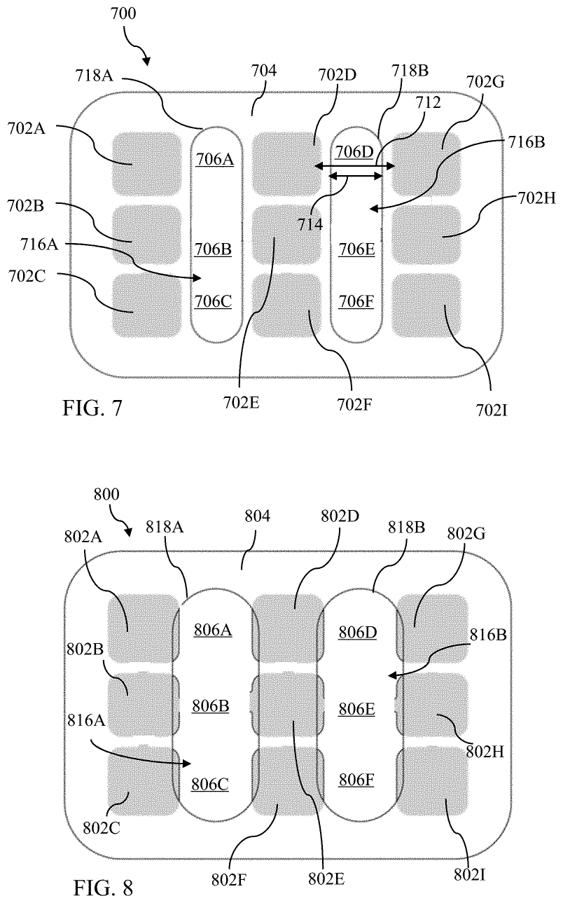

FIGS. 4-10 illustrate examples of transducer apparatuses that may be used to apply TTFields to a subject's body. Each example transducer apparatus includes a substrate having an adhesive layer and one or more non-adhesive regions (or void regions) where no adhesive is present. The non-adhesive regions leave portions of the subject's skin uncovered with respect to adhesive while the transducer is attached to the subject's skin and used to apply TTFields to a target region of the subject's body. Positioning a non-adhesive region over an area of the subject's skin, which may have previously been in contact with adhesive, allows this area of the subject's skin to “breathe” and/or recover from the prior contact it had with adhesive. As some subjects experience skin irritation in response to prolonged interaction of the skin with adhesive, slightly shifting the transducer so that a non-adhesive region is positioned over an affected area of the subject's skin may help to minimize, reduce, or prevent irritation of the subject's skin throughout TTField treatment while maintaining the transducer in an optimal or near optimal location on the subject's body. In the embodiments of FIGS. 4-6, transducers (400, 500, 600) may be repositioned in this manner via rotation of the transducer (400, 500, 600) about a centroid (e.g., 530 as shown in FIG. 5) of the electrode array. In the embodiments of FIGS. 7-10, transducers (700, 800, 900, 1000) may be repositioned via translation of the transducer (700, 800, 900, 1000).

FIGS. 4-10 depict example transducer apparatuses (400, 500, 600, 700, 800, 900, 1000) in accordance with the present disclosure. The transducer apparatus (400, 500, 600, 700, 800, 900, 1000) may include an array of electrodes (402A-F, 502A-F, 602A-F, 702A-I, 802A-I, 902A-I, 1002A-I). The array of electrodes (402A-F, 502A-F, 602A-F, 702A-I, 802A-I, 902A-I, 1002A-I) is configured to be positioned over the subject's body with a face of the array facing the subject's body. FIGS. 4-10 illustrate the transducer apparatus as viewed from a direction perpendicular to this face of the array.

In each of FIGS. 4-10, the transducer apparatus (400, 500, 600, 700, 800, 900, 1000) includes a substrate comprising an adhesive layer (404, 504, 604, 704, 804, 904, 1004A-C) for attaching the transducer apparatus to the subject's body. A non-adhesive region (406A-F, 506A-F, 606A-G, 706A-F, 806A-F, 906A-F, 1006A-F), where no adhesive layer is present, is located between a pair of adjacent electrodes of the array. As shown in FIGS. 4, 7, and 10, the non-adhesive region (e.g., 406E, 706D, 1006C) spans at least 25% of a total distance (412, 712, 1012) between the pair of adjacent electrodes for at least one measurement (414, 714, 1014) as measured along a straight line between the pair of adjacent electrodes. This distance spanned by the non-adhesive region compared to the distance between the corresponding pair of electrodes is present in each of FIGS. 4-10, though only explicitly drawn on FIGS. 4, 7, and 10. In some embodiments, the non-adhesive region spans at least 50% of the total distance between the pair of adjacent electrodes. In some embodiments, the non-adhesive region spans at least 100% of the total distance between the pair of adjacent electrodes (e.g., as shown in FIGS. 5, 6, 8, and 9). In some embodiments, the straight line between the pair of adjacent electrodes is drawn from a centroid of one electrode to the centroid of the adjacent electrode, and the measurement of the distance between the two adjacent electrodes is made from the peripheral edge of one electrode to the peripheral edge of the adjacent electrode on the line connecting the two centroids.

In each of FIGS. 4-10, a ratio Sₐ/Sₑ of a surface area of the at least one adhesive region (404, 504, 604, 704, 804, 904, 1004) (Sₐ) to a surface area of the array of electrodes (402A-F, 502A-F, 602A-F, 702A-I, 802A-I, 902A-I, 1002A-I) (Sₑ) is less than 1.5. The surface area of the at least one adhesive region (Sₐ) is defined as all adhesive portions of the transducer apparatus (400, 500, 600, 700, 800, 900, 1000) that touch the subject's skin upon application of the transducer apparatus to the subjects body, and excludes any areas of the adhesive region overlapping the electrodes (also PCB and connector) of the array. In some embodiments, the ratio Sₐ/Sₑ is less than 1.3 (e.g., as shown in FIGS. 4, 5, and 10). In some embodiments, the ratio Sₐ/Sₑ is less than 1.0 (e.g., as shown in FIGS. 4 and 10). These surface area ratios Sₐ/Sₑ are much lower than those of conventional transducer apparatuses, which generally include large areas of adhesive entirely surrounding the electrode array. Transducer apparatuses having lower surface area ratios Sₐ/Sₑ as disclosed herein may be used with electrodes made from polymer films. The lighter weight and thinness of these electrodes may allow the array to be adequately attached to the subject's skin using less adhesive. Using less adhesive to attach the transducer apparatus to the subject's skin leaves more of the subject's skin uncovered by the adhesive, thereby reducing or minimizing an amount of irritation of the skin that may occur.

The electrodes in presently disclosed transducers may have any desired shape. In some embodiments, one or more of the electrodes may have a triangular shape, a substantially triangular shape with rounded corners, a truncated triangular shape, a substantially truncated triangular shape with rounded corners, a wedge shape, a substantially wedge shape with rounded corners, a truncated wedge shape, or a substantially truncated wedge shape with rounded corners. For example, the electrodes (402A-F, 502A-F, 602A-F) in FIGS. 4-6 are substantially wedge shaped with rounded corners. In other embodiments, one or more of the electrodes may have a square, rectangular, or hexagonal shape or a substantially square, rectangular, or hexagonal shape with one or more rounded corners. For example, the electrodes (702A-I, 802A-I, 902A-I, 1002A-I) in FIGS. 7-10 are substantially square shaped with rounded corners or rectangular shaped with rounded corners. As shown in FIGS. 4-6, individual electrodes (402A-F, 502A-F, 602A-F) may be electrically coupled together via one or more PCB layer(s) (405, 505, 605) or wire(s). Although FIGS. 7-10 do not depict PCB layer(s) or wire(s), it should be understood that the electrodes therein are electrically coupled together to output a desired electrical field.

In FIGS. 4-6, the transducer (400, 500, 600) includes six electrodes (402, 502, 602), while in FIGS. 7-10 the transducer (700, 800, 900, 1000) includes nine electrodes. Other embodiments may include different numbers of electrodes (e.g., 2, 3, 4, 5, 7, 8, 10, 11, 12, or more electrodes). In FIGS. 4-5 and 7-10, the transducer (400, 500, 700, 800, 900, 1000) includes six non-adhesive regions (406, 506, 706, 806, 906, 1006) between adjacent electrodes, while in FIG. 6 the transducer (600) includes seven non-adhesive regions (606) between adjacent electrodes. Other embodiments may include different numbers of non-adhesive regions (e.g., 1, 2, 3, 4, 5, 8, 9, 10, 11, 12, or more non-adhesive regions).

The substrate of disclosed transducers may have any desired shape. For example, the substrate may have a substantially square or rectangular shape, or a substantially square or rectangular shape with rounded corners (e.g., as shown in FIGS. 7-10). In another example, the substrate may have a substantially circular, oval, ovoid, ovaloid, or elliptical shape (e.g., as shown in FIGS. 5 and 6). As another example, the substrate may have an irregular shape (e.g., as shown in FIG. 4).

In the transducer 400 of FIG. 4, an external perimeter 408 of the adhesive layer 404 has multiple concave portions 410A-E, and non-adhesive regions 406A-E (or void regions) are bounded by (or defined by) the concave portions 410A-E, respectively. Each of these non-adhesive regions 406A-E spans at least 25% of a total distance between two corresponding pairs of adjacent electrodes on either side of the non-adhesive region for at least one measurement as measured along a straight line between the pair of adjacent electrodes. In addition, an opening 416 is present within the adhesive layer 404 (e.g., at the center of the array). The opening 416 has a perimeter 418 surrounded on all sides by the adhesive layer 404, and another non-adhesive region 406F is located within the perimeter 418 of the opening 416.

In the transducer 500 of FIG. 5, multiple openings 516A-E are present within the adhesive layer 504, and each of the openings 516A-E have perimeters 518A-E, respectively, surrounded on all sides by the adhesive layer 504. Non-adhesive regions 506A-E are located within the perimeters of the openings 516A-E, respectively. Each of these non-adhesive regions 506A-E spans at least 25% (e.g., 100% as shown) of a total distance between two corresponding pairs of adjacent electrodes on either side of the non-adhesive region for at least one measurement as measured along a straight line between the pair of adjacent electrodes. In addition, an external perimeter 508 of the adhesive layer 504 has a concave portion 510, and a non-adhesive region 506F (or void region) is bounded by (or defined by) the concave portion 510 of the external perimeter 508. Non-adhesive region 506F spans at least 25% (e.g., 100% as shown) of a distance between the pair of adjacent electrodes 502F, 502A on either side of the non-adhesive region for at least one measurement as measured along a straight line between the pair of adjacent electrodes.

In the transducer 600 of FIG. 6, an external perimeter 608 of the adhesive layer 604 has a concave portion 610, and non-adhesive regions 606A-G are each bounded by the same concave portion 610 of the external perimeter 608. Each of these non-adhesive regions 606A-G spans 100% of a total distance between two corresponding pairs of adjacent electrodes on either side of the non-adhesive region. This is because the adhesive layer 604 extends substantially around the outer periphery of the array, leaving a large opening 616 at the center of the array.

In each of the transducers (700, 800) of FIGS. 7 and 8, two openings (716A/716B, 816A/816B) are present within the adhesive layer (704, 804), each opening located between two adjacent rows of electrodes. The openings (716A/716B, 816A/816B) each have perimeters (718A/718B, 818A/818B) surrounded on all sides by the adhesive layer (704, 804). Non-adhesive regions (706A-C, 806A-C) are located within the perimeter of the first opening (716A, 816A), and non-adhesive regions (706D-F, 806D-F) are located within the perimeter of the second opening (716B, 816B). Each non-adhesive region (706A-F, 806A-F) spans at least 25% of a total distance between pairs of adjacent electrodes on either side of the non-adhesive region.

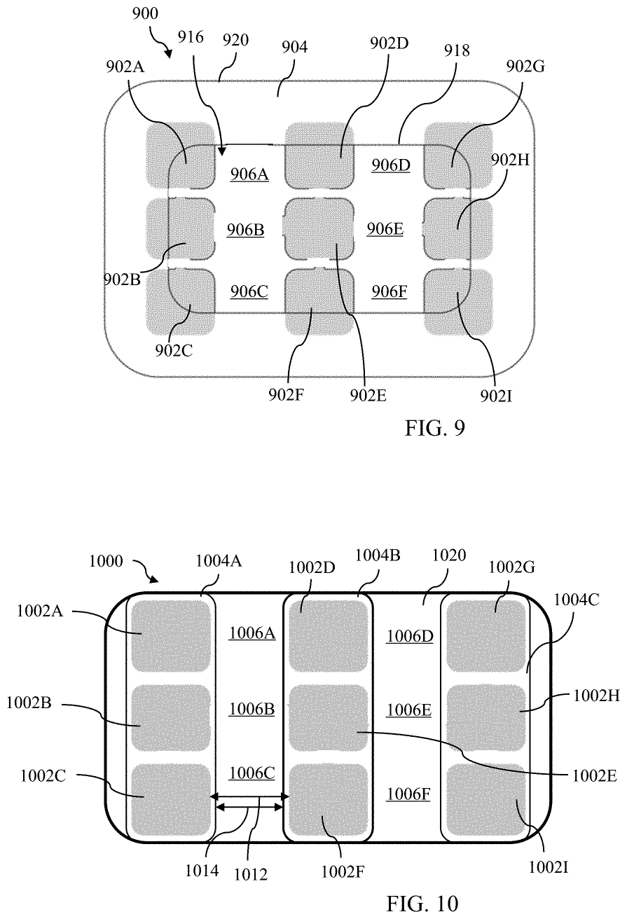

In the transducer 900 of FIG. 9, an opening 916 is present within the adhesive layer 904 (e.g., at the center of the array). The opening 916 has a perimeter 918 surrounded on all sides by the adhesive layer 904, and non-adhesive regions 906A-F are each bounded by the perimeter 918 of the opening 916. Each of these non-adhesive regions 906A-F spans 100% of a total distance between two corresponding pairs of adjacent electrodes on either side of the non-adhesive region. This is because the adhesive layer 904 extends substantially around the outer periphery of the array while leaving a large opening at the center of the array. As shown in FIG. 9, one or more electrodes (e.g., center electrode 902E) may not overlap the adhesive layer 904 at any portion thereof. That is, the one or more electrodes (e.g., 902E) do not have any adhesive thereon and do not directly contact an adhesive layer. Other electrodes (e.g., 902A-D and 902F-I) may partially overlap the adhesive layer, as shown. In other embodiments, multiple electrodes may not overlap the adhesive layer such that the multiple electrodes are located completely in a void region.

Additional details present in one or more of FIGS. 4-9 will now be provided. In each of FIGS. 5 and 7-9, the substrate (illustrated as adhesive layer 504, 704, 804, 904) has at least one internal opening (516, 716, 816, 916) therein, this opening being defined by an internal edge (e.g., perimeter 518, 718, 818, 918) of the substrate and surrounded on all sides by the adhesive layer (504, 704, 804, 904). Moreover, a void region having no adhesive layer is defined by the internal opening (516, 716, 816, 916). At least part of the void region spans 25% or more of a total distance (e.g., 712) between two adjacent electrodes for at least one measurement (e.g., 714) as measured along a straight line between the two adjacent electrodes.

In FIGS. 5, 8, and 9, at least one edge of each of the two adjacent electrodes (e.g., 502A and 502B in FIG. 5) is located in the void region defined by the perimeter (518A). In FIG. 7, on the other hand, the void region defined by the perimeter 718 does not overlap any of the electrodes 702A-I in the array.

As shown in FIGS. 4, 5, and 7-9, the void regions may have different shapes. For example, the void region may have a triangular shape, a substantially triangular shape with rounded corners, a truncated triangular shape, a substantially truncated triangular shape with rounded corners, a wedge shape, a substantially wedge shape with rounded corners, a truncated wedge shape, or a substantially truncated wedge shape with rounded corners (e.g., as in FIG. 5). As another example, the void region may have an elongated shape with two parallel edges extending between longitudinal ends of the void region, as in FIGS. 7-9. The void region may have a substantially square or rectangular shape, or substantially square or rectangular shape with rounded corners, as in FIGS. 7-9. In other embodiments, the void region may have a circular, oval, ovoid, ovaloid, or elliptical shape (e.g., region 406F of FIG. 4). This may also be the case, for example, in a transducer similar to the transducer 600 of FIG. 6 having edges of the adhesive layer 604 defined by two (closed) circles. In still other embodiments, the void region may have an irregular shape (e.g., region 506C of FIG. 5).

The number of void regions may also differ. For example, as in FIGS. 5, 7, and 8, the transducer (500, 700, 800) may include multiple void regions having no adhesive layer and defined by internal openings 516A-E, 716A-B, 816A-B) in the substrate. As another example, in the transducer 900 of FIG. 9, the void region defined by opening 916 is the only void region having no adhesive layer. As shown in FIG. 5, one or more void regions (e.g., non-adhesive regions 506A-E) defined by internal openings 516A-E may be combined with one or more other void regions (e.g., non-adhesive region 506F) defined by a substantially concave shaped inward facing edge (e.g., concave portion 510).

In each of FIGS. 6 and 9, the adhesive layer (604, 904) has a substantially convex shaped outward facing edge (620, 920), and a void region (e.g., opening 616, 916) having no adhesive therein is defined by a substantially concave shaped inward facing edge (610, 918) of the adhesive layer, the void region overlapping at least a portion of each electrode (602A-F, 902A-I) in the array. In some embodiments, the void region may have a substantially circular, oval, ovoid, ovaloid or elliptical shape, as in FIG. 6. In other embodiments, the void region may have a substantially square or rectangular shape, or a substantially square or rectangular shape with rounded corners, as in FIG. 9. In still other embodiments, the void region may have an irregular shape. The void region may be open or closed. For example, as in FIG. 9, the inward facing edge 918 of the adhesive layer 904 may be entirely separate from the outward facing edge 920 of the adhesive layer. As in FIG. 6, the inward facing edge 610 of the adhesive layer 604 may be continuous with the outward facing edge 620 of the adhesive layer.

FIG. 10 illustrates a transducer 1000 having multiple adhesive regions. In the transducer 1000, the substrate has at least two separate adhesive layers 1004 spaced apart from each other by a non-adhesive region 1006. A first adhesive region 1004A covers at least a first electrode 1002C of the array, and a second adhesive region 1004B covers at least a second electrode 1002F of the array. A void space (e.g., non-adhesive region 1006C) having no adhesive is located between the first and second adhesive regions 1004A and 1004B, and a distance 1014 between the first and second adhesive regions 1004A and 1004B is at least 25% of a total distance 1012 between a first electrode 1002C and a second electrode 1002F for at least one measurement as measured along a straight line between the electrodes. As illustrated, the first adhesive region 1004A may additionally cover electrode 1002B, and the second adhesive region 1004B may additionally cover electrode 1002E. A void space (e.g., non-adhesive region 1006B) having no adhesive is located between the first and second adhesive regions 1004A and 1004B, and a distance between the first and second adhesive regions 1004A and 1004B is at least 25% of a total distance between the electrode 1002B and the electrode 1002E for at least one measurement as measured along a straight line between the electrodes. The substrate of transducer 1000 may include a bandage 1020 with the first and second adhesive regions 1004A and 1004B thereon. As illustrated, the transducer 1000 may include three adhesive regions 1004A-C. Other embodiments may include other numbers of adhesive regions on the substrate.

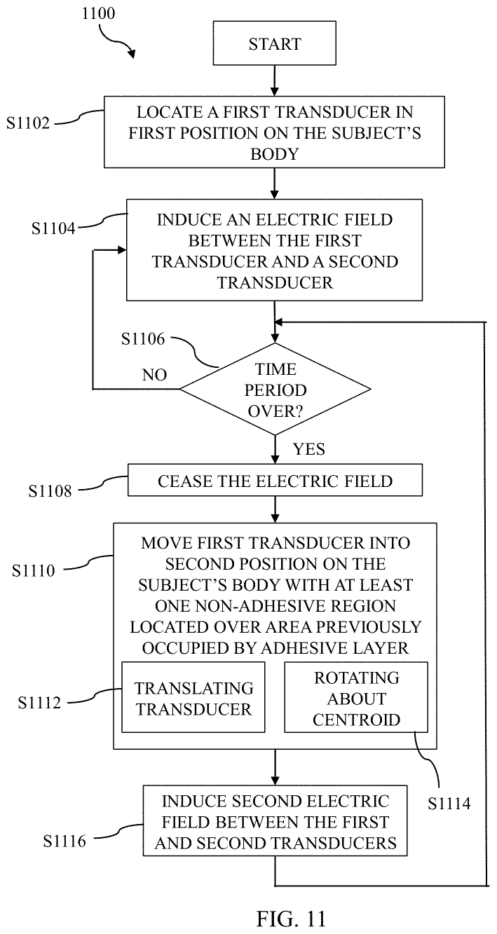

FIG. 11 depicts an example method 1100 of applying TTFields to a subject's body in accordance with the present techniques. The method 1100 begins at step S1102 with locating a first transducer in a first position on the subject's body. The first transducer includes a substrate having an adhesive layer that couples the first transducer to the subject's body and a plurality of electrodes arranged on the substrate. A non-adhesive region, where no adhesive layer is present, is located between two adjacent electrodes of the first transducer.

At step S1104, the method 1100 includes inducing an electric field between the first transducer and a second transducer located on the subject's body. At step S1106, the method 1100 includes determining whether a first period of time has passed. After inducing the electric field for more than the first period of time, the method 1100 proceeds to step S1108, which includes ceasing the electric field.

At step S1110, the method 1100 includes moving the first transducer into a second position on the subject's body, wherein in the second position the non-adhesive region in the substrate is located over an area that was previously occupied by at least a portion of the adhesive layer. This movement may involve, for example, translating (S1112) the first transducer with respect to a surface of the subject's body; or rotating (S1114) the first transducer about its centroid. At step S1116, the method 1100 may include inducing a second electric field between the first transducer and the second transducer. After step S1116, the method 1100 may repeat through steps S1106 to S1116 until the end of a TTFields treatment.

The invention includes the following illustrative embodiments.

Embodiment 1: A transducer apparatus for delivering tumor treating fields to a subject's body, the transducer apparatus comprising: an array of electrodes, the array configured to be positioned over the subject's body with a face of the array facing the subject's body; a substrate comprising an adhesive layer for attaching the transducer apparatus to the subject's body, wherein: the array of electrodes is disposed entirely on a first side of the substrate, the face of the array faces away from the substrate, the adhesive layer is on the first side of the substrate facing the same direction as the face of the array; and wherein, when viewed from a direction perpendicular to the face of the array, a non-adhesive region, where no adhesive layer is present, is located between a pair of adjacent electrodes of the array, wherein the non-adhesive region spans at least 25% of a total distance between the pair of adjacent electrodes for at least one measurement as measured along a straight line between the pair of adjacent electrodes.

Embodiment 2: The transducer apparatus of Embodiment 1, wherein the non-adhesive region spans at least 50% of the total distance between the pair of adjacent electrodes.

Embodiment 3: The transducer apparatus of Embodiment 1, wherein the non-adhesive region spans at least 100% of the total distance between the pair of adjacent electrodes.

Embodiment 4: The transducer apparatus of Embodiment 1, wherein, when viewed from the direction perpendicular to the face of the array, an external perimeter of the adhesive layer has at least one concave portion; and the non-adhesive region is bounded by a concave portion of the at least one concave portion of the external perimeter.

Embodiment 5: The transducer apparatus of Embodiment 4, wherein, when viewed from the direction perpendicular to the face of the array, a second non-adhesive region, where no adhesive layer is present, is located between a second pair of adjacent electrodes of the array, wherein the second non-adhesive region spans at least 25% of a total distance between the second pair of adjacent electrodes for at least one measurement as measured along a straight line between the second pair of adjacent electrodes; and the second non-adhesive region is also bounded by the concave portion of the at least one concave portion of the external perimeter.

Embodiment 6: The transducer apparatus of Embodiment 4, wherein, when viewed from the direction perpendicular to the face of the array, a second non-adhesive region, where no adhesive layer is present, is located between a second pair of adjacent electrodes of the array, wherein the second non-adhesive region spans at least 25% of a total distance between the second pair of adjacent electrodes for at least one measurement as measured along a straight line between the second pair of adjacent electrodes; the external perimeter of the adhesive layer has a second concave portion; and the second non-adhesive region is bounded by the second concave portion of the at least one concave portion of the external perimeter.

Embodiment 7: The transducer apparatus of Embodiment 1, wherein when viewed in the direction perpendicular to the face of the array, an opening is present within the adhesive layer, the opening having a perimeter surrounded on all sides by the adhesive layer; and the non-adhesive region is located within the perimeter of the opening.

Embodiment 8: The transducer apparatus of Embodiment 7, wherein, when viewed from the direction perpendicular to the face of the array, a second non-adhesive region, where no adhesive layer is present, is located between a second pair of adjacent electrodes of the array, wherein the second non-adhesive region spans at least 25% of a total distance between the second pair of adjacent electrodes for at least one measurement as measured along a straight line between the second pair of adjacent electrodes; and the second non-adhesive region is also located within the perimeter of the opening.

Embodiment 9: The transducer apparatus of Embodiment 7, wherein, when viewed from the direction perpendicular to the face of the array, a second non-adhesive region, where no adhesive layer is present, is located between a second pair of adjacent electrodes of the array, wherein the second non-adhesive region spans at least 25% of a total distance between the second pair of adjacent electrodes for at least one measurement as measured along a straight line between the second pair of adjacent electrodes; and a second opening is present within the adhesive layer, the second opening having a perimeter surrounded on all sides by the adhesive layer; and the second non-adhesive region is located within the perimeter of the second opening.

Embodiment 10: The transducer apparatus of Embodiment 1, wherein when viewed from the direction perpendicular to the face of the array, the substrate has at least two separate adhesive layers spaced apart from each other by the non-adhesive region.

Embodiment 11: The transducer apparatus of Embodiment 1, wherein the non-adhesive region is a space where no portion of the substrate is located.

Embodiment 12: The transducer apparatus of Embodiment 1, wherein the non-adhesive region is a region of the substrate that does not have the adhesive layer.

Embodiment 13: The transducer apparatus of Embodiment 1, wherein one or more of the electrodes in the array have a square, rectangular, or hexagonal shape or a substantially square, rectangular, or hexagonal shape with one or more rounded corners.

Embodiment 14: The transducer apparatus of Embodiment 1, wherein one or more of the electrodes of the array have a triangular shape, a substantially triangular shape with rounded corners, a truncated triangular shape, a substantially truncated triangular shape with rounded corners, a wedge shape, a substantially wedge shape with rounded corners, a truncated wedge shape, or a substantially truncated wedge shape with rounded corners.

Embodiment 15: The transducer apparatus of Embodiment 1, wherein the electrodes in the array comprise polymer films.

Embodiment 16: The transducer apparatus of Embodiment 1, wherein the electrodes in the array comprise ceramic electrodes.

Embodiment 17: A method of applying tumor treating fields to a subject's body, the method comprising: locating a first transducer in a first position on the subject's body, the first transducer comprising: an array of electrodes, the array configured to be positioned over the subject's body with a face of the array facing the subject's body; a substrate comprising an adhesive layer for attaching the transducer apparatus to the subject's body, wherein: the array of electrodes is disposed entirely on a first side of the substrate, the face of the array faces away from the substrate, the adhesive layer is on the first side of the substrate facing the same direction as the face of the array; and wherein, when viewed from a direction perpendicular to the face of the array, a non-adhesive region, where no adhesive layer is present, is located between a pair of adjacent electrodes of the array, wherein the non-adhesive region spans at least 25% of a total distance between the pair of adjacent electrodes for at least one measurement as measured along a straight line between the pair of adjacent electrodes.

Embodiment 18: The method of Embodiment 17, wherein, when viewed from the direction perpendicular to the face of the array, an external perimeter of the adhesive layer has at least one concave portion; and the non-adhesive region is bounded by a concave portion of the at least one concave portion of the external perimeter.

Embodiment 19: The method of Embodiment 17, wherein, when viewed from the direction perpendicular to the face of the array, a second non-adhesive region, where no adhesive layer is present, is located between a second pair of adjacent electrodes of the array, wherein the second non-adhesive region spans at least 25% of a total distance between the second pair of adjacent electrodes for at least one measurement as measured along a straight line between the second pair of adjacent electrodes; the external perimeter of the adhesive layer has a second concave portion; and the second non-adhesive region is bounded by the second concave portion of the at least one concave portion of the external perimeter.

Embodiment 20: A transducer apparatus for delivering tumor treating fields to a subject's body, the transducer apparatus comprising: an array of electrodes, the array configured to be positioned over the subject's body with a face of the array facing the subject's body; a substrate with at least one adhesive region thereon for attaching the transducer apparatus to the subject's body; wherein, when viewed from a direction perpendicular to the face of the array, a ratio Sₐ/Sₑ of a surface area of the at least one adhesive region (Sₐ) to a surface area of the array of electrodes (Sₑ) is less than 1.5, wherein the surface area of the at least one adhesive region (Sₐ) is defined as all adhesive portions of the transducer apparatus that touch the subject's skin upon application of the transducer apparatus to the subjects body, and excludes any areas of the adhesive region overlapping the electrodes of the array.

Embodiment 21: The transducer apparatus of Embodiment 20, wherein the ratio Sₐ/Sₑ is less than 1.3.

Embodiment 22: The transducer apparatus of Embodiment 20, wherein the ratio Sₐ/Sₑ is less than 1.0.

Embodiment 23: A transducer apparatus for delivering tumor treating fields to a subject's body, the transducer apparatus comprising: an array of electrodes, the array configured to be positioned over the subject's body with a face of the array facing the subject's body; a substrate comprising an adhesive layer for attaching the transducer apparatus to the subject's body, wherein: the array of electrodes is disposed entirely on a first side of the substrate, the face of the array faces away from the substrate, the adhesive layer is on the first side of the substrate facing the same direction as the face of the array; wherein, when viewed from a direction perpendicular to the face of the array, the substrate has an internal opening therein, the internal opening being defined by an internal edge of the substrate and surrounded on all sides by the adhesive layer; a void region having no adhesive layer is defined by the internal opening; at least part of the void region is located between two adjacent electrodes of the array; and the at least part of the void region spans 25% or more of a total distance between the two adjacent electrodes for at least one measurement as measured along a straight line between the two adjacent electrodes.

Embodiment 24: The transducer apparatus of Embodiment 23, wherein when viewed from the direction perpendicular to the face of the array, the void region does not overlap any of the electrodes in the array.

Embodiment 25: The transducer apparatus of Embodiment 23, wherein when viewed from the direction perpendicular to the face of the array, at least one edge of each of the two adjacent electrodes is located in the void region.

Embodiment 26: The transducer apparatus of Embodiment 23, wherein the void region has a triangular shape, a substantially triangular shape with rounded corners, a truncated triangular shape, a substantially truncated triangular shape with rounded corners, a wedge shape, a substantially wedge shape with rounded corners, a truncated wedge shape, or a substantially truncated wedge shape with rounded corners.

Embodiment 27: The transducer apparatus of Embodiment 23, wherein the void region has an elongated shape with two parallel edges extending between longitudinal ends of the void region.

Embodiment 28: The transducer apparatus of Embodiment 23, wherein the void region has a substantially square or rectangular shape, or substantially square or rectangular shape with rounded corners.

Embodiment 29: The transducer apparatus of Embodiment 23, wherein the void region has a circular, oval, ovoid, ovaloid, or elliptical shape.

Embodiment 30: The transducer apparatus of Embodiment 23, further comprising: a second void region defined by a substantially concave shaped inward facing edge of the adhesive layer, the second void region having no adhesive therein, wherein at least part of the second void region is located between two other adjacent electrodes of the array.

Embodiment 31: The transducer apparatus of Embodiment 23, wherein the void region is one of multiple void regions having no adhesive layer and defined by internal openings in the substrate.

Embodiment 32: The transducer apparatus of Embodiment 23, wherein the void region is the only void region having no adhesive layer and defined by an internal opening in the substrate.

Embodiment 33: The transducer apparatus of Embodiment 23, wherein the substrate has a substantially square or rectangular shape, or substantially square or rectangular shape with rounded corners.

Embodiment 34: The transducer apparatus of Embodiment 23, wherein the substrate has a substantially circular, oval, ovoid, ovaloid, or elliptical shape.

Embodiment 35: A transducer apparatus for delivering tumor treating fields to a subject's body, the transducer apparatus comprising: an array of electrodes, the array configured to be positioned over the subject's body with a face of the array facing the subject's body; a substrate comprising an adhesive layer for attaching the transducer apparatus to the subject's body, wherein: the array of electrodes is disposed entirely on a first side of the substrate, the face of the array faces away from the substrate, the adhesive layer is on the first side of the substrate facing the same direction as the face of the array; wherein, when viewed from a direction perpendicular to the face of the array, the adhesive layer has a substantially convex shaped outward facing edge; a void region is defined by a substantially concave shaped inward facing edge of the adhesive layer, the void region having no adhesive therein; and the void region overlapping at least a portion of each electrode in the array.

Embodiment 36: The transducer apparatus of Embodiment 35, wherein the void region has a substantially circular, oval, ovoid, ovaloid, or elliptical shape.

Embodiment 37: The transducer apparatus of Embodiment 35, wherein the void region has a substantially square or rectangular shape, or substantially square or rectangular shape with rounded corners.

Embodiment 38: The transducer apparatus of Embodiment 35, wherein the inward facing edge of the adhesive layer is entirely separate from the outward facing edge of the adhesive layer.

Embodiment 39: The transducer apparatus of Embodiment 35, wherein the inward facing edge of the adhesive layer is continuous with the outward facing edge of the adhesive layer.

Embodiment 40: A transducer apparatus for delivering tumor treating fields to a subject's body, the transducer apparatus comprising: an array of electrodes, the array configured to be positioned over the subject's body with a face of the array facing the subject's body; a substrate comprising an adhesive layer for attaching the transducer apparatus to the subject's body, wherein: the array of electrodes is disposed entirely on a first side of the substrate, the face of the array faces away from the substrate, the adhesive layer is on the first side of the substrate facing the same direction as the face of the array; wherein, when viewed from a direction perpendicular to the face of the array, an outer perimeter of the adhesive layer has a concave portion; a void region having no adhesive layer is defined by the concave portion; and the void region is located between and spans at least 25% of a total distance between two adjacent electrodes of the array for at least one measurement as measured along a straight line between the two adjacent electrodes of the array.

Embodiment 41: The transducer apparatus of Embodiment 40, wherein the void region is substantially wedge shaped, or substantially wedge shaped with rounded corner(s).

Embodiment 42: The transducer apparatus of Embodiment 40, wherein when viewed from a direction perpendicular to the face of the array, the outer perimeter of the adhesive layer has a second concave portion; a second void region having no adhesive layer is defined by the second concave portion; and the second void region is located between and spans at least 25% of a total distance between another two adjacent electrodes of the array for at least one measurement as measured along a straight line between the two adjacent electrodes of the array.

Embodiment 43: The transducer apparatus of Embodiment 40, wherein: the substrate has an internal opening therein, the internal opening being defined by an internal edge of the substrate and surrounded on all sides by the adhesive layer; a second void region having no adhesive layer is defined by the internal opening; and at least part of the second void region is located between another two adjacent electrodes of the array.

Embodiment 44: A transducer apparatus for delivering tumor treating fields to a subject's body, the transducer apparatus comprising: an array of electrodes, the array configured to be positioned over the subject's body with a face of the array facing the subject's body; a substrate with multiple adhesive regions thereon for attaching the transducer apparatus to the subject's body, wherein: the array of electrodes is disposed entirely on a first side of the substrate, the face of the array faces away from the substrate, the adhesive regions are on the first side of the substrate facing the same direction as the face of the array; wherein, when viewed from a direction perpendicular to the face of the array, a first adhesive region covers at least a first electrode of the array; a second adhesive region covers at least a second electrode of the array; a void space having no adhesive is located between the first and second adhesive regions; and a distance between the first and second adhesive regions is at least 25% of a total distance between the first electrode and the second electrode for at least one measurement as measured along a straight line between the first electrode and the second electrode.

Embodiment 45: The transducer apparatus of Embodiment 44, wherein when viewed from the direction perpendicular to the face of the array, the first adhesive region covers a third electrode of the array; the second adhesive region covers a fourth electrode of the array; and the distance between the first and second adhesive regions is at least 25% of a total distance between the third electrode and the fourth electrode for at least one measurement as measured along a straight line between the third electrode and the fourth electrode.

Embodiment 46: The transducer apparatus of Embodiment 44, wherein the substrate comprises a bandage with the first and second adhesive regions.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A transducer apparatus for delivering tumor treating fields to a subject's body, the transducer apparatus comprising:
an array of electrodes, the array configured to be positioned over the subject's body with a face of the array facing the subject's body;
a substrate comprising an adhesive layer for attaching the transducer apparatus to the subject's body, wherein:
the array of electrodes is disposed entirely on a first side of the substrate,
the face of the array faces away from the substrate,
the adhesive layer is on the first side of the substrate facing the same direction as the face of the array; and
wherein, when viewed from a direction perpendicular to the face of the array,
a non-adhesive region, where no adhesive layer is present, is located between a pair of adjacent electrodes of the array, wherein the non-adhesive region spans at least 25% of a total distance between the pair of adjacent electrodes for at least one measurement as measured along a straight line between the pair of adjacent electrodes.

2. The transducer apparatus of claim 1, wherein the non-adhesive region spans at least 50% of the total distance between the pair of adjacent electrodes.

3. The transducer apparatus of claim 1, wherein the non-adhesive region spans at least 100% of the total distance between the pair of adjacent electrodes.

4. The transducer apparatus of claim 1, wherein, when viewed from the direction perpendicular to the face of the array,
an external perimeter of the adhesive layer has at least one concave portion; and
the non-adhesive region is bounded by a concave portion of the at least one concave portion of the external perimeter.

5. The transducer apparatus of claim 4, wherein, when viewed from the direction perpendicular to the face of the array,
a second non-adhesive region, where no adhesive layer is present, is located between a second pair of adjacent electrodes of the array, wherein the second non-adhesive region spans at least 25% of a total distance between the second pair of adjacent electrodes for at least one measurement as measured along a straight line between the second pair of adjacent electrodes; and
the second non-adhesive region is also bounded by the concave portion of the at least one concave portion of the external perimeter.

6. The transducer apparatus of claim 4, wherein, when viewed from the direction perpendicular to the face of the array,
a second non-adhesive region, where no adhesive layer is present, is located between a second pair of adjacent electrodes of the array, wherein the second non-adhesive region spans at least 25% of a total distance between the second pair of adjacent electrodes for at least one measurement as measured along a straight line between the second pair of adjacent electrodes;
the external perimeter of the adhesive layer has a second concave portion; and
the second non-adhesive region is bounded by the second concave portion of the at least one concave portion of the external perimeter.

7. The transducer apparatus of claim 1, wherein when viewed in the direction perpendicular to the face of the array,
an opening is present within the adhesive layer, the opening having a perimeter surrounded on all sides by the adhesive layer; and
the non-adhesive region is located within the perimeter of the opening.

8. The transducer apparatus of claim 7, wherein, when viewed from the direction perpendicular to the face of the array,
a second non-adhesive region, where no adhesive layer is present, is located between a second pair of adjacent electrodes of the array, wherein the second non-adhesive region spans at least 25% of a total distance between the second pair of adjacent electrodes for at least one measurement as measured along a straight line between the second pair of adjacent electrodes; and
the second non-adhesive region is also located within the perimeter of the opening.

9. The transducer apparatus of claim 7, wherein, when viewed from the direction perpendicular to the face of the array,
a second non-adhesive region, where no adhesive layer is present, is located between a second pair of adjacent electrodes of the array, wherein the second non-adhesive region spans at least 25% of a total distance between the second pair of adjacent electrodes for at least one measurement as measured along a straight line between the second pair of adjacent electrodes; and
a second opening is present within the adhesive layer, the second opening having a perimeter surrounded on all sides by the adhesive layer; and
the second non-adhesive region is located within the perimeter of the second opening.

10. The transducer apparatus of claim 1, wherein when viewed from the direction perpendicular to the face of the array, the substrate has at least two separate adhesive layers spaced apart from each other by the non-adhesive region.

11. The transducer apparatus of claim 1, wherein the non-adhesive region is a space where no portion of the substrate is located.

12. The transducer apparatus of claim 1, wherein the non-adhesive region is a region of the substrate that does not have the adhesive layer.

13. The transducer apparatus of claim 1, wherein one or more of the electrodes in the array have a square, rectangular, or hexagonal shape or a substantially square, rectangular, or hexagonal shape with one or more rounded corners.

14. The transducer apparatus of claim 1, wherein one or more of the electrodes of the array have a triangular shape, a substantially triangular shape with rounded corners, a truncated triangular shape, a substantially truncated triangular shape with rounded corners, a wedge shape, a substantially wedge shape with rounded corners, a truncated wedge shape, or a substantially truncated wedge shape with rounded corners.

15. The transducer apparatus of claim 1, wherein the electrodes in the array comprise polymer films.

16. The transducer apparatus of claim 1, wherein the electrodes in the array comprise ceramic electrodes.

17. A method of applying tumor treating fields to a subject's body, the method comprising:
locating a first transducer in a first position on the subject's body, the first transducer comprising:
an array of electrodes, the array configured to be positioned over the subject's body with a face of the array facing the subject's body;
a substrate comprising an adhesive layer for attaching the first transducer to the subject's body, wherein:
the array of electrodes is disposed entirely on a first side of the substrate,
the face of the array faces away from the substrate,
the adhesive layer is on the first side of the substrate facing the same direction as the face of the array; and
wherein, when viewed from a direction perpendicular to the face of the array, a non-adhesive region, where no adhesive layer is present, is located between a pair of adjacent electrodes of the array, wherein the non-adhesive region spans at least 25% of a total distance between the pair of adjacent electrodes for at least one measurement as measured along a straight line between the pair of adjacent electrodes;
inducing an electric field between the first transducer and a second transducer located on the subject's body.

18. The method of claim 17, wherein, when viewed from the direction perpendicular to the face of the array,
an external perimeter of the adhesive layer has at least one concave portion; and
the non-adhesive region is bounded by a concave portion of the at least one concave portion of the external perimeter.

19. The method of claim 17, wherein, when viewed from the direction perpendicular to the face of the array,
a second non-adhesive region, where no adhesive layer is present, is located between a second pair of adjacent electrodes of the array, wherein the second non-adhesive region spans at least 25% of a total distance between the second pair of adjacent electrodes for at least one measurement as measured along a straight line between the second pair of adjacent electrodes;
the external perimeter of the adhesive layer has a second concave portion; and
the second non-adhesive region is bounded by the second concave portion of the at least one concave portion of the external perimeter.

20. A transducer apparatus for delivering tumor treating fields to a subject's body, the transducer apparatus comprising:
an array of electrodes, the array configured to be positioned over the subject's body with a face of the array facing the subject's body;
a substrate with at least one adhesive region thereon for attaching the transducer apparatus to the subject's body;
wherein, when viewed from a direction perpendicular to the face of the array,
a ratio Sₐ/Sₑ of a surface area of the at least one adhesive region (Sₐ) to a surface area of the array of electrodes (Sₑ) is less than 1.5, wherein the surface area of the at least one adhesive region (Sₐ) is defined as all adhesive portions of the transducer apparatus that touch the subject's skin upon application of the transducer apparatus to the subjects body, and excludes any areas of the adhesive region overlapping the electrodes of the array.
